(19) United States
(12) Patent Application Publication
(10) Pub. No.: US 2016/0367708 A1
(43) Pub. Date: Dec. 22, 2016

(54) COMPOSITIONS AND METHODS OF TREATING CANCER

(71) Applicant: THE JOHNS HOPKINS UNIVERSITY, Baltimore, MD (US)

(72) Inventors: John Martin Abraham, Woodbine, MD (US); Yulan Cheng, Ellicott City, MD (US); Martin G. Pomper, Baltimore, MD (US); Stephen J. Meltzer, Lutherville, MD (US); Joseph Herman, Perry Hall, MD (US)

(73) Assignee: THE JOHNS HOPKINS UNIVERSITY, Baltimore, MD (US)

(21) Appl. No.: 14/901,301

(22) PCT Filed: Jun. 27, 2014
(86) PCT No.: PCT/US2014/044604

§ 371 Date: Dec. 28, 2015

Related U.S. Application Data
(60) Provisional application No. 61/840,771, filed on Jun. 28, 2013.

(51) Int. Cl. A61K 51/12; A61K 9/00

(52) U.S. Cl. CPC A61K 51/1217; A61K 9/0019

(57) ABSTRACT

The present invention relates to the field of cancer. More specifically, the present invention provides compositions and methods for treating cancer using aqueous ³²P. In certain embodiments, the present invention provides a method for treating cancer in a patient comprising the step of intravenously administering a low dose of aqueous ³²P monophosphate or ³²P pyrophosphate to the patient.

### CROSS-REFERENCE TO RELATED APPLICATIONS

This application claims the benefit of U.S. Provisional Application No. 61/840,771, filed Jun. 28, 2013, which is incorporated herein by reference in its entirety.

### STATEMENT OF GOVERNMENTAL INTEREST

This invention was made with government support under grant no. IROICA133012 and grant no. P50CA062924 awarded by the National Institutes of Health. The government has certain rights in the invention.

### FIELD OF THE INVENTION

The present invention relates to the field of cancer. More specifically, the present invention provides compositions and methods for treating cancer using aqueous ³²P.

### BACKGROUND OF THE INVENTION

Mammalian cancer cells are efficiently killed by beta particles emitted from radionuclides. For example, the ability of ¹³I to home to thyroid tissue has been exploited for decades as a therapeutic strategy against thyroid cancer and Graves' disease, and this strategy is still used in over 50% of thyroid cancer patients in the United States. Similarly, ¹³¹I-Bexxar and ⁹⁰Y-Zevalin are used to treat non-Hodgkin's lymphoma based on antibodies against the CD20 cell surface antigen. Moreover, ⁹⁰Y-radiolabeled somatostatin is also utilized to treat neuroendocrine tumors. [³²P]ATP emits electrons with an energy level intermediate between that of ⁹⁰Y and ¹³¹I; these electrons have a path length of up to 5 mm in tissues. Thus, each electron can penetrate thousands of cells. The resulting cross-fire results in a “bystander effect” which greatly amplifies the killing power of each ³²P atom located in or near a tumor. In addition, ³²P has a much longer half-life than ⁹⁰Y or ¹³I: this is an advantage, since radioactivity levels in tumors do not diminish as rapidly from natural decay.

Anti-cancer therapeutics are often assessed by their ability to inhibit xenografted tumors in nude mice. Successful drugs tested in this fashion have included Rituxan and other antibodies directed against cell surface molecules. Examples of drugs designed against broader targets include the anti-VEGF antibody Avastin, which inhibits the establishment of the tumor microenvironment, and cisplatin, which targets rapidly growing cells. Small molecules present many advantages as anti-cancer therapeutics, such as better tumor penetration and low immunogenicity.

The search for novel therapeutic agents that are effective against cancer has been difficult and expensive. The activity of anti-cancer candidate agents against human cancer-derived cell lines in immunocompromised mice has been a valuable tool in this research. Because ATP is a naturally-occurring small molecule, its radiolabeled form poses many advantages as a potential anti-cancer therapeutic agent.

The use of ³²P to combat various types of cancer has been attempted since the 1930s, with disappointing results. We have conducted extensive literature searches on the use of ³²P as a potential anti-cancer agent during the past fifty years. In these published papers, the injected form of ³²P is virtually always some type of a colloidal suspension wherein ³²P is part of a particulate. This practice has been performed because the ³²P is usually injected directly into the primary tumor, and the colloidal suspension prevents the isotope from leaving the intended target at the point of injection and spreading throughout the patient.

Inorganic ³²P has been used for decades as a therapeutic agent in human polycythemia vera and essential thrombocythemia. ³²P is in a simple aqueous form that travels rapidly throughout the body and is rapidly absorbed by rapidly proliferating bone marrow cells, where it successfully eliminates the overactive cells. Parenthetically, inorganic ³²P has also been used for palliation of bone pain in metastatic cancer patients.

### SUMMARY OF THE INVENTION

The present invention is based, at least in part, on the discovery that the use of an aqueous form of ³²P represents a tremendous advance in the use of ³²P as an effective anti-cancer therapeutic agent. We have found that, in a syngeneic mouse model system, the intravenous injection of a relatively small amount of aqueous ³²P in different simple forms results in rapid and significant inhibition of established mouse tumors. Using a cell culture assay, it has been shown that the uptake of the ³²P radioisotope into the cellular DNA is far more efficient in killing cells than that obtained when the cells are merely bombarded with equivalent amounts of electrons external to the cell. In addition, it has been shown that this much higher cell killing efficiency is obtained by a mechanism of causing double-strand DNA breaks that is unique to the ³²P isotope and cannot be utilized by other beta-emitting radioisotopes such as ¹³¹I and ⁹⁰Y.

Accordingly, in one aspect, the present invention provides methods for treating cancer. In one embodiment, a method for treating cancer in a patient comprises the step of systemically administering to the patient an effective amount of aqueous ³²P. In a specific embodiment, ³²P is ³²P monophosphate. In another specific embodiment, the ³²P is ³²P pyrophosphate. In certain embodiments, the aqueous ³²P is administered intravenously.

In another specific embodiment, the patient has metastatic cancer. In yet another embodiment, the cancer is colon cancer. In fact, the cancer can be any cancer including, but not limited to, lung, breast or pancreatic cancer.

In particular embodiments, the ³²P is administered in a low dose amount. In one embodiment, the ³²P is administered in a dosage range of about 0.5 mCi to about 10.0 mCi. In another embodiment, the ³²P is administered in a dosage range of about 0.75 mCi to about 7.5 mCi. In a specific embodiment, the ³²P is administered in a dose of about 750 μCi.

In certain embodiments, the present invention provides a method for treating cancer in a patient comprising the step of intravenously administering a low dose of aqueous ³²P monophosphate or ³²P pyrophosphate to the patient. In a specific embodiment, the patient has metastatic cancer. In another embodiment, the cancer is colon cancer. In other embodiments, the cancer is lung, breast or pancreatic cancer. The ³²P can be administered in a dosage range of about 0.75 mCi to about 7.5 mCi. In a specific embodiment, the ³²P is administered in a dose of about 750 μCi.

### BRIEF DESCRIPTION OF THE FIGURES

FIG. 1. A cell proliferation assay using WST-1 was performed to assess the relative cell killing ability of ³²Pmonophosphate that is taken up by cells into the DNA versus that which is not taken up by the cells, but subjected only to electron bombardment.

FIG. 2. Inhibition of cell growth by [³²P]PO₄ or [⁹⁰Y]. The WST-1 proliferation assay was done to determine the level of cell kill by ³²P or by ⁹⁰Y. BALB/c tumor CRL2836 cells or HeLa cells were exposed to 0 Ci, 1 μCi, 2.5 μCi, or 5 μCi in complete medium. After 24 hours, the radioactivity was removed and cells were grown in non-radioactive complete medium. WST-1 cell proliferation assays were done at Days 1, 2, 3, 4, and 5 and all assays were done in triplicate. The mean is shown plus/minus the standard deviation. The student's two-sided t-test determined the P value shown.

FIG. 3. An experiment similar to that shown in FIG. 1 was conducted using chamber slides that compared the presence of double-strand DNA breaks in cells that incorporated the radioisotope versus cells that were only exposed to the electron bombardment from ³²P isotope contained in tubes.

FIG. 4. Determination of double-strand DNA breaks in cells caused by exposure to [³²P]PO₄ or ⁹⁰Y. HeLa cells or mouse BALB/c CRL2836 cells were grown in multiple sections of chamber slides and exposed to 0 μCi or 3 μCi of [³²P]PO₄ or ⁹⁰Y in complete medium at Day 0. After 24 hours, the radioactivity was removed and cells were grown in non-radioactive complete medium. At Day 1, 2, or 3 the presence of double-strand DNA breaks in the cells was determined by staining for phosphorylated H2-AX histones which indicate double-strand DNA damage.

FIG. 5. Inhibition of BALB/c syngeneic tumor growth by [³²P]PO₄. Syngeneic BALB/c CRL2836 tumors were established in the rear flanks of BALB/c mice at Day 0. After ten days during which the tumors became well vascularized, mice received an injection of 5 μCi of [³²P]PO₄ intra-venously via the tail vein. The tumor volumes are shown as the mean of six tumors plus/minus the standard error of the mean. The student's two-sided t-test determined the P value shown.

FIG. 6. Model of double-strand DNA break after incorporation of [³²P]PO₄. The radioisotope is incorporated into the ribose-phosphate backbone of DNA in dividing cells. The process of decaying to sulfur (³²S) breaks the backbone bond of the initial strand at a 67% rate and releases a high energy beta particle (electron) that must only travel two nm across the helix to the opposite target strand. Although an emitted electron that travels in the perfect orientation from this ³²P decay to sever the opposite strand will only occur at a low percentage of the time, it is still much higher and more efficient than those electrons which are generated by other beta-producing radioisotopes on the cell surface or in the cytosol that must travel distances that are usually one thousand times or more longer in length.

FIG. 7. ³²P is directly incorporated into cellular DNA. Mouse CRL2836 or human HeLa cell lines were seeded into cell culture plates and grown for 24 h (is defined as Day 0). Cells were then either incubated overnight with ³²P[PO₄], grown for 2 d in non-radioactive medium and the DNA extracted (3 Days), or grown for 24 h, incubated with ³²P[PO₄] for 24 h, grown for 24 h in non-radioactive medium and the DNA extracted (2 Days), or grown for 48 h, incubated with ³²P[PO₄] for 24 hours, washed with complete medium and the nucleic acid extracted (1 Day). The extracted nucleic acids were incubated for two hours with or without DNase I, and the digestion products were run on a 5% polyacrylamide gel, exposed to film for 24 h, and developed. More than half of the ³²P retained by the cells that were incubated with ³²P[PO₄]for 24 h and then grown in non-radioactive medium for 48 h before the DNA was extracted had been permanently incorporated into cellular DNA.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Radioisotopes that emit beta particles, such as radioiodine, can effectively kill target cells. An aqueous form of ³²P has been used for decades to treat noncancerous human myeloproliferative diseases in which too many platelets or red blood cells are produced. A colloidal form of ³²P (chromic phosphate) has been tried for several decades to fight human cancer with mixed and often disappointing results. We have discovered that a number of simple aqueous forms (compounds) of ³²P are extremely effective in inhibiting the growth of tumors in several different mouse models.

One of the key novel aspects of this invention is the use of AQUEOUS solutions of simple molecules that contain ³²P to find and kill the target cancer cells. Past conventional use of ³²P as an anti-cancer drug utilized colloidal preparations that were injected directly into the tumor which would prevent the radioisotope from spreading. We have found that a single IV injection of the aqueous form of ³²P can result in this agent being incorporated directly into the DNA of the target cells. In addition, we have elucidated the mechanism of cell death that the use of ³²P facilitates. Unlike other radioactive and efficient target cell killers such as radioiodine which target the DNA from outside of the nucleus, the incorporation of the ³²P isotope directly into the DNA results in a novel and more efficient method of double-strand DNA breakage which results in the death of the cell.

### I. Definitions

The following definitions are used throughout this specification. Other definitions are embedded within the specification for ease of reference.

As used herein, the term “cancer” means a type of hyperproliferative disease that includes a malignancy characterized by deregulated or uncontrolled cell growth. Cancers of virtually every tissue are known. Examples of cancer include, but are not limited to, carcinoma, lymphoma, blastoma, sarcoma, and leukemia or lymphoid malignancies. More particular examples of such cancers are noted below and include squamous cell cancer (e.g., epithelial squamous cell cancer), lung cancer (including small-cell lung cancer, non-small cell lung cancer, adenocarcinoma of the lung and squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer, uterine carcinoma, salivary gland carcinoma, kidney or renal cancer, prostate cancer, thyroid cancer, hepatic carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

The term “cancer,” is encompassed within the scope of the broader term “abnormal cellular proliferation, which can also be referred to as “excessive cellular proliferation or “cellular proliferative disease.” Examples of diseases associated abnormal cellular proliferation include metastatic tumors, malignant tumors, benign tumors, cancers, pre-cancers, hyperplasias, warts, and polyps, as well as non-cancerous conditions such as benign melanomas, benign chondroma, benign prostatic hyperplasia, moles, dysplastic nevi, dysplasia, hyperplasias, and other cellular growths occurring within the epidermal layers. Classes of precancers include acquired small or microscopic precancers, acquired large lesions with nuclear atypia, precursor lesions occurring with inherited hyperplastic syndromes that progress to cancer, and acquired diffuse hyperplasias and diffuse metaplasias. Examples of small or microscopic precancers include HGSIL (high grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

As used herein, the term “effective,” means adequate to accomplish a desired, expected, or intended result. More particularly, an “effective amount” or a “therapeutically effective amount” is used interchangeably and refers to an amount of an AT-1 modulator, perhaps in further combination with yet another therapeutic agent, necessary to provide the desired “treatment” (defined herein) or therapeutic effect, e.g., an amount that is effective to prevent, alleviate, treat or ameliorate symptoms of a disease or prolong the survival of the subject being treated. In particular embodiments, the pharmaceutical compositions of the present invention are administered in a therapeutically effective amount to treat patients suffering from an AT-1-mediated disease, disorder or condition (e.g., a disease, disorder or condition associated with an autism spectrum disorder or a disorder in which neurogenesis is impaired). As would be appreciated by one of ordinary skill in the art, the exact low dose amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

### II. Pharmaceutical Compositions and Administration

Accordingly, a pharmaceutical composition of the present invention may comprise an effective amount of ³²P including ³²P monophosphate and ³²P pyrophosphate. As used herein, the term “effective,” means adequate to accomplish a desired, expected, or intended result. More particularly, an “effective amount” or a “therapeutically effective amount” is used interchangeably and refers to an amount of ³²P, perhaps in further combination with yet another therapeutic agent, necessary to provide the desired “treatment” (defined herein) or therapeutic effect, e.g., an amount that is effective to prevent, alleviate, treat or ameliorate symptoms of a cancer or prolong the survival of the subject being treated. In particular embodiments, the pharmaceutical compositions of the present invention are administered in a therapeutically effective amount to treat patients suffering from cancer. As would be appreciated by one of ordinary skill in the art, the exact low dose amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The pharmaceutical compositions of the present invention are in biologically compatible form suitable for administration in vivo for subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which ³²P is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions.

In general, the pharmaceutical compositions comprising ³²P may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., pharmaceutical compositions comprising ³²P, optionally in combination with another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effect is the therapeutic index and it may be expressed as the ratio LD50/ED50. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Previous regimens of inorganic ³²P for polycythemia vera and essential thrombocythemia used very high doses of ³²P, in the range of 15-30 mCi. In contrast, the present invention utilizes a low dose amount, in general, about an order of magnitude lower. Thus in certain embodiments, the low dose amount of ³²P is about 0.1 mCi to about 10 mCi. The dose can range from about 0.2 mCi to about 9 mCi, about 0.5 mCi to about 8.5 mCi, about 1.0 mCi to about 8.0 mCi, about 1.5 mCi to about 7.5 mCi, about 2.0 mCi to about 7.0 mCi, about 2.5 mCi to about 6.5 mCi, about 3.0 mCi to about 6.0 mCi, about 3.5 mCi to about 5.5 mCi, or about 4.0 mCi to about 5.0 mCi.

More specifically, the aqueous ³²P may comprise a dose of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 and/or 10.0 mCi. Those of skill in the art will recognize that the precise quantity of such a compound to be administered will vary from case to case, and is best determined by a skilled practitioner such as a physician.

In other embodiments, the low dose amount of ³²P is about 10 mCi to about 15 mCi, including 10 mCi, 11 mCi, 12 mCi, 13 mCi, and 14 mCi. More specifically, the aqueous ³²P may comprise a dose of about 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9 and/or 15.0 mCi. In a specific embodiment, the low dose is no more than 15 mCi.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

The pharmaceutical compositions may further be combined with one or more additional therapeutic agents. A combination therapy regimen may be additive, or it may produce synergistic results (e.g., in a particular disease, greater than expected for the combined use of the two agents).

The compositions can be administered simultaneously or sequentially by the same or different routes of administration. The determination of the identity and amount of the pharmaceutical compositions for use in the methods of the present invention can be readily made by ordinarily skilled medical practitioners using standard techniques known in the art. In specific embodiments, ³²P of the present invention can be administered in combination with an effective amount of another therapeutic agent. In particular embodiments, the other therapeutic agent can be another treatment for cancer.

In various embodiments, the ³²P of the present invention in combination with an another therapeutic agent (e.g., an anti-cancer therapeutic) may be administered at about the same time, less than 1 minute apart, less than 2 minutes apart, less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patent visit.

In certain embodiments, the ³²P of the present invention in combination with another therapeutic agent are cyclically administered. Cycling therapy involves the administration of a first therapy (e.g., the ³²P) for a period of time, followed by the administration of a second therapy (e.g., another therapeutic agent) for a period of time, optionally, followed by the administration of perhaps a third therapy for a period of time and so forth, and repeating this sequential administration, e.g., the cycle, in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### The Killing of Target Cells by DNA Incorporation of ³²P Utilizes a Unique Mechanism of Inducing Double-Strand Breaks

Radioisotopes that emit beta particles, such as radioiodine, can effectively kill target cells. An aqueous form of ³²P has been used for decades to treat non-cancerous human myeloproliferative diseases in which too many platelets or red blood cells are produced. A colloidal form of ³²P (chromic phosphate) has been tried for several decades to fight human cancer with mixed and often disappointing results. We have discovered that a number of simple aqueous forms (compounds) of ³²P are extremely effective in inhibiting the growth of tumors in several different mouse models. The incorporation of ³²P into cellular DNA results in an extremely efficient method of causing double-strand breaks that cannot be accomplished by other radioisotopes that emit electrons.

### Materials and Methods

Study Design. The overall objective of this study was to evaluate the potential of the elemental radioisotope ³²P as a future possible anti-cancer drug. This report contains results from in vitro cell culture assays and from an in vivo syngeneic mouse cancer system. Cell proliferation experiments used two different cell lines and the inhibition of cell growth was measured by the WST-1 proliferation assay. In addition, detection of double-strand DNA breaks caused by ³²P versus the more powerful beta-emitter ⁹⁰Y was done by using an antibody which detects the phosphorylation of the H2AX histone. Finally, the ability of a single intravenous low dose of ³²P to significantly inhibit the growth of tumor cells in a BALB/c syngeneic tumor model was demonstrated.

Measurement of In Vitro Cell Killing by ³²P Radioisotope. Two thousand cells of the mouse BALB/c CRL2836 cell line or the human HeLa cell line were growing in complete medium in wells of a 96-well plate and were exposed to either 0 uCi, 3 uCi or 10 uCi of the [³²P]pyrophosphate or the [³²P]monophosphate radioisotopes. One set of triplicate wells contained medium with the designated form of the isotope, while another set of triplicate wells had the isotope contained in small tubes placed in the wells. This radioisotope in the tube resulted in these cells being exposed to the emitted electrons, but not actually taking up the isotope into the cell. After a 24 hour exposure time, fresh non-radioactive medium was added and WST-1 proliferation assays were done at several subsequent time points.

Measurement of in vitro cell killing by ³²P and ⁹⁰Y. Two thousand cells of the mouse BALB/c CRL2836 cell line or the human HeLa cell line were grown in complete medium in a 96-well plate and at Day 0 were exposed to either 0, 1, 2.5, or 5 μCi of ⁹⁰Y radioisotope or the [³²P]PO₄ radioisotope in complete medium. After a 24 h incubation, the radioisotope-containing medium was removed and replaced with non-radioactive complete medium (Day 1). WST-1 proliferation assays (Roche Applied Science, Indianapolis, Ind.) were done on Days 1, 2, 3, 4, or 5 to directly measure cell growth. Each experiment was performed in triplicate.

Assessment of Double-Strand DNA Breaks by ³²P. Ten thousand HeLa cells were seeded in Lab-TekII chamber slides. At day 0, cells were treated with 10 uCi ³²P either in the tube or added directly into the medium and at day 1 fresh non-radioactive medium was added. At Day 1, Day 2, or Day 3, cells were fixed with 10% formalin at room temperature for 10 minutes, washed with PBS for two minutes, and 0.2% Triton X-100 with 10% FBS in PBS was added for 15 minutes for permeabilization. After a rinse with PBS, primary mouse anti-human H2AX antibody with 1:1000 dilution (Millipore) was incubated for 1 hr at RT, and washed with PBS for 5 minutes for two times. A dilution of 1:400 goat anti-mouse IgG with Alexa Fluor (Molecular probe, Life Technology) was incubated for 1 hour at RT, washed with PBS for 5 minutes for two times. Cells were stained with Hoechst solution (1:1000) for counter staining.

Assessment of Double-Strand DNA Breaks by ³²P and ⁹⁰Y. Ten thousand HeLa cells were seeded in Lab-TekII chamber slides (Thermo Fisher Scientific Inc., Waltham, Mass.). At Day 0 cells were treated with 0 or 3 μCi of ³²P or ⁹⁰Y in complete medium. At Day 1, all wells were gently washed and fresh non-radioactive medium was added. At Day 1, Day 2, or Day 3, cells were fixed with 10% formalin at room temperature for 10 min, washed with PBS for two min, and permeabilized with 0.2% Triton X-100 with 10% FBS in PBS for 15 min. After a rinse with PBS, primary mouse anti-human H2AX antibody with 1:1000 dilution (Millipore, Billerica, Mass.) was incubated for 1 h at ambient temperature, and washed twice with PBS for 5 min. A dilution of 1:400 goat anti-mouse IgG with Alexa Fluor (Life Technologies, Grand Island, N.Y.) was incubated for 1 h at ambient temperature and washed twice with PBS for 5 min. Cells were stained with Hoechst solution (1:1000).

Assessement of ³²P incorporated into DNA. One hundred and fifty thousand mouse CRL2836 or human HeLa cells were seeded into a six-well cell culture plate and grown for 24 h (defined as Day 0). Cells were then either incubated overnight with ³²P[PO₄], grown for 2 d in non-radioactive medium and the DNA extracted (3 Days), or grown for 24 h, incubated with ³²P[PO₄] for 24 h, grown for 24 h in non-radioactive medium and the DNA extracted (2 Days), or grown for 48 h, incubated with ³²P[PO₄] for 24 hours, washed with complete medium and the nucleic acid extracted (1 Day). Nucleic acid was extracted using the DNAeasy Blood and Tissue Kit (Qiagen, Valencia, Calif.) and aliquots were incubated with or without four units of DNase I (New England Biolabs, Ipswich, Mass.) for two h at 37° C. before the samples were run on a 5% acrylamide gel, exposed to film overnight at 4° C. and developed.

Establishment of mouse tumors. Syngeneic BALB/c mouse tumors were established by injecting 2×10⁶ BALB/c tumor CRL2836 cells (American Type Cell Culture, Manassas, Va.) in a volume of 0.2 mL (50% Matrigel, 50% 1×PBS) subcutaneously in the left rear and right rear flank. All mice were female, 10 weeks of age, and purchased from Charles River Laboratories (Wilmington, Mass.).

³²P-Mediated tumor growth inhibition. After ten days, during which time well-vascularized tumors were established, an injection of 5 μCi of the monophosphate form of ³²P (Perkin-Elmer, Cat. # NEX06000) was injected intravenously via the tail vein in 0.1 mL of 1×HBSS. Six tumors (three animals) were studied in each group. After injection, tumor growth was measured three times per week with a digital caliper and the volume was determined using the formula: volume=½(width)²×(length).

Statistical analysis. The data from the WST-1 cell proliferation are presented as means±standard deviation and the significance was determined using the unpaired Student's t test. The tumor volumes of the untreated and treated mice are shown as means±SE and no outliers were excluded for any reason. The significance was determined using the unpaired Student's t test.

### Results

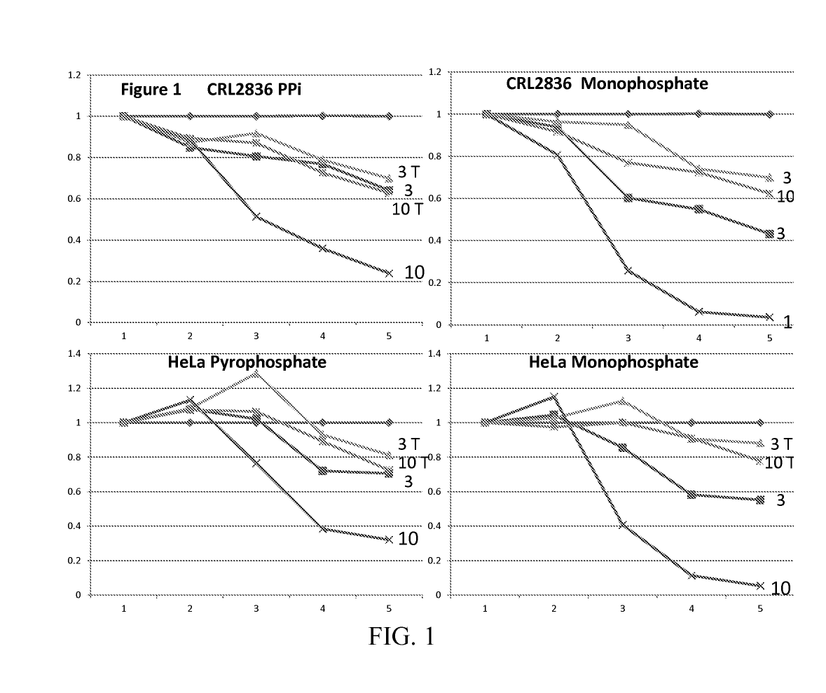

To assess the relative cell killing ability of [³²P]monophosphate that is taken up by cells into the DNA versus that of is not taken up by the cells, but subjected only to electron bombardment, a cell proliferation assay using WST-1 was undertaken (FIG. 1). The [³²P]monophosphate was added directly to the cell culture medium for 1 day and then replaced with non-radioactive medium and, over a period of up to six days, the cell proliferation was directly compared to identical cell cultures in which the [³²P]monophosphate remained in tubes placed in the wells. The cells that incorporated the radioisotope had far greater levels of cell death as monitored by the lack of cell growth.

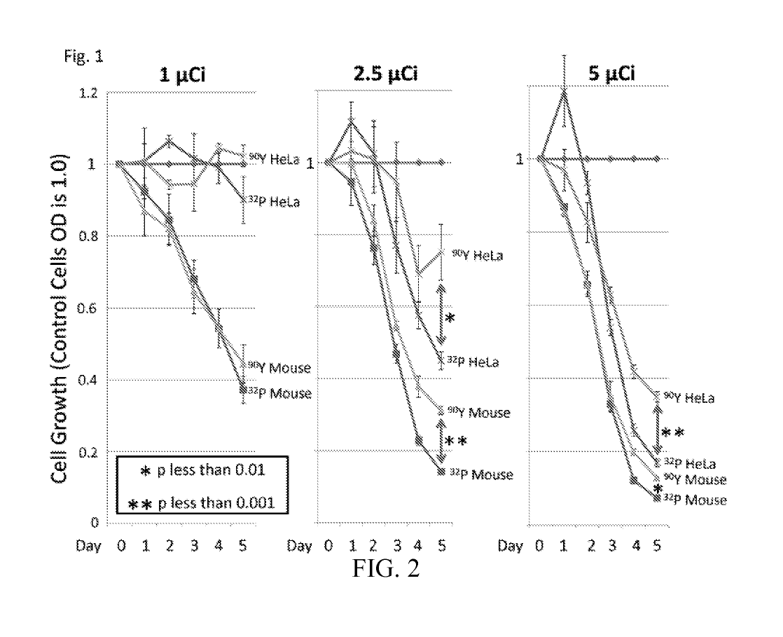

In a similar experiment, cells exposed to [³²P]PO₄ were compared with those exposed to identical amounts of the more powerful beta-particle emitter, ⁹⁰Y, after which WST-1 cell proliferation assays were performed (FIG. 2). Different cell lines are expected to demonstrate varying levels of susceptibility to radioisotopes. The 1 μCi dose showed that HeLa cells were less susceptible to beta-emitting isotopes than were BALB/c mouse CRL2836 cells, which originated as an osteosarcoma and were isolated after it had metastasized to lung. Both the 2.5 μCi and 5 μCi doses demonstrated similar results in both cell lines. Although ⁹⁰Y would have been expected to be more lethal than ³²P based on its higher-energy electrons, ³²P killed cells more efficiently than did ⁹⁰Y. In comparisons of the 2.5 μCi and 5 μCi doses, [³²P]PO₄ effected survival rates at Day 5 that were barely half of those produced by ⁹⁰Y.

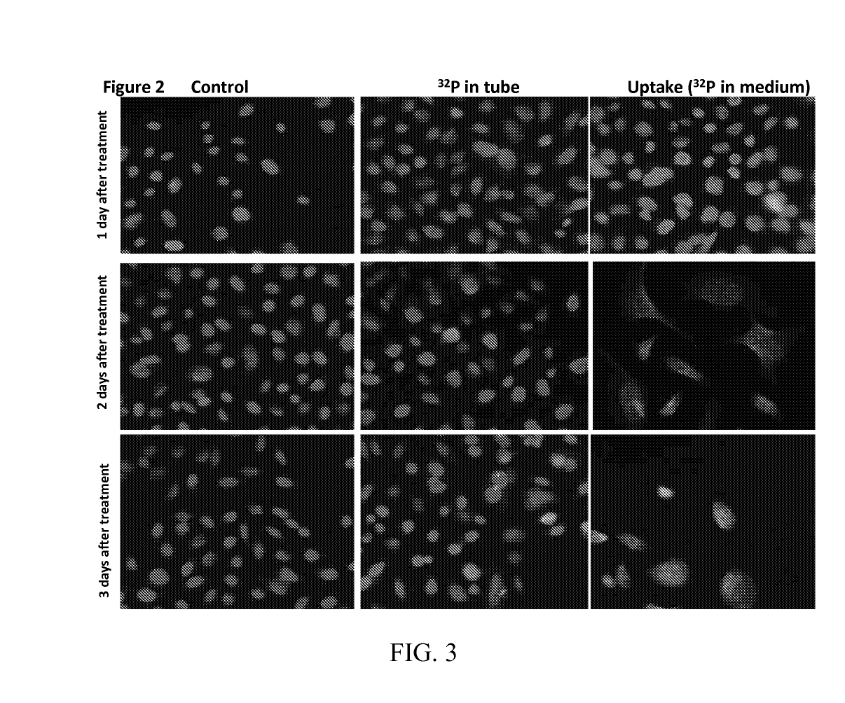

Another experiment was conducted using chamber slides that compared the presence of double-strand DNA breaks in cells that incorporated the radioisotope versus cells that were only exposed to the electron bombardment from ³²P isotope contained in tubes (FIG. 3). The level of double-strand DNA breaks was determined by an immunostain assay of histone H2-AX phosphorylation that detects these breaks in both strands of DNA at the same location.

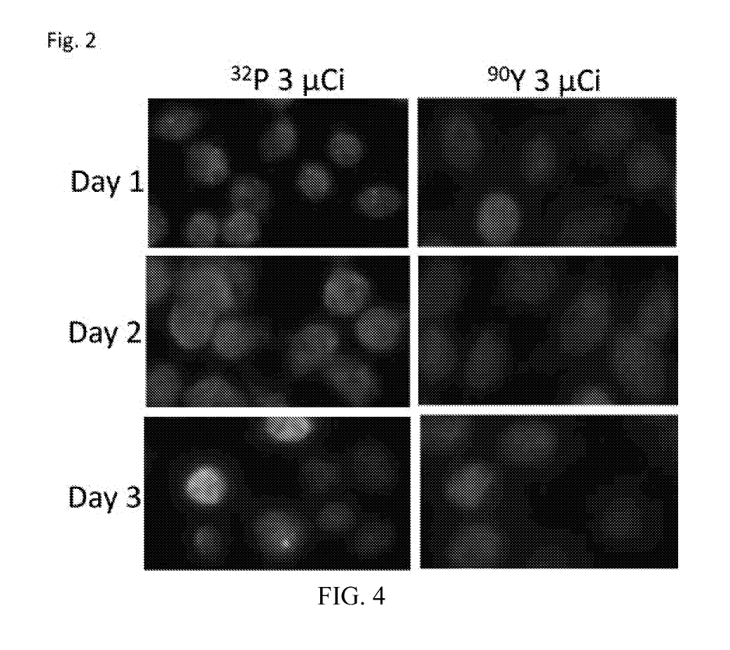

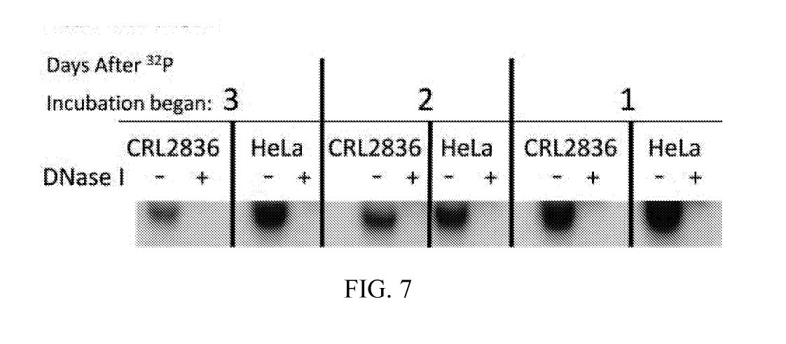

The H2AX assay was used to compare double-strand DNA breakage in cells incubated with ³²P vs. ⁹⁰Y (FIG. 4). This assay accurately detects breakage in both strands of DNA at the same genomic locus. Nuclear staining of HeLa cells demonstrated significant, time-dependent double-strand DNA breakage in cells exposed to ³²P, while those exposed to identical levels of ⁹⁰Y-based radiation had substantially less or no detectable DNA double-strand breakage. Digestion with DNase I demonstrated that ³²P was directly incorporated into cellular DNA (FIG. 7).

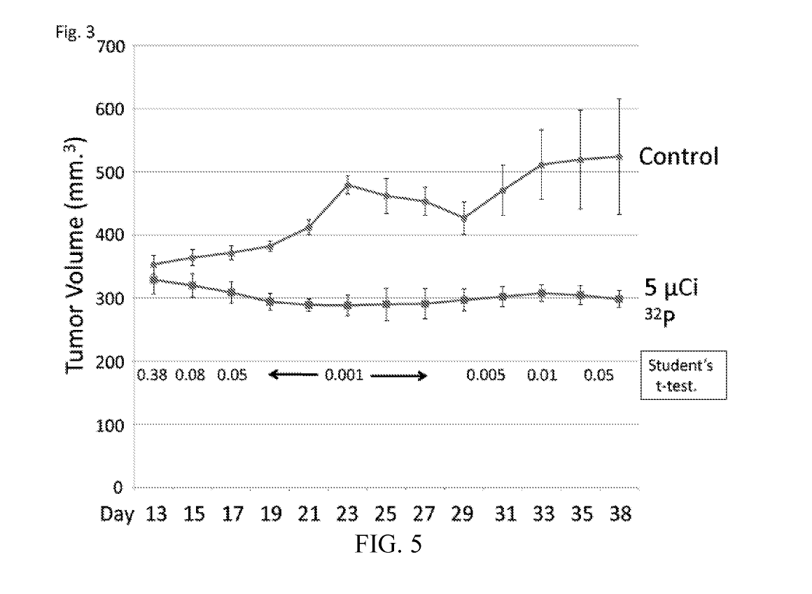

Previously, we demonstrated significant inhibition of HeLa cell xenografts in nude mice by a single low-dose intravenous (IV) injection of [³²P]ATP. Here, we chose syngeneic BALB/c mice to more closely recapitulate human malignancy. A single IV injection of aqueous [³²P]PO₄ significantly inhibited established syngenic tumor growth in BALB/c mice (FIG. 5). Importantly, there were no apparent detrimental effects of [³²P]PO₄ on the health of the mice, either in terms of weight gain or overall activity levels.

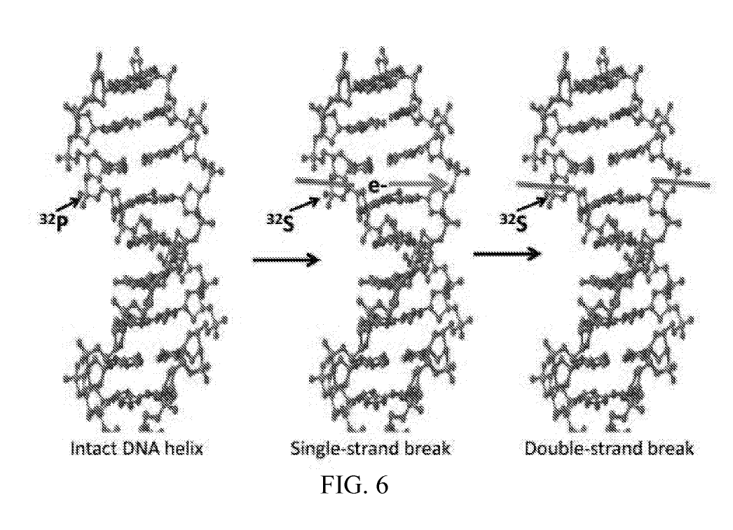

FIG. 6 depicts a proposed mechanism for ³²P-induced cell killing. In this schematic, ³²P is incorporated directly into one strand of replicating DNA. Radioactive decay of ³²P to ³²S causes chemical breakage of that same DNA strand. Next, the electron released by this decay event travels only two nm to reach the opposite strand of the double helix, severing it and causing a double-strand break at this genomic locus. This mechanism stands in stark contrast to non-incorporated beta-emitting radioisotopes, where only a small fraction of emitted electrons travel in the precise orientation necessary to strike the opposite strand and cause a double-strand DNA break. With ³²P, close proximity of the contralateral target strand makes this double-strand breakage much more likely to occur.

### Discussion

Aqueous [32P]PO4 offers many potential advantages over other anti-cancer therapeutic agents. Firstly, it allows for rapid systemic distribution and incorporation by both primary tumors and metastases. Moreover, 32P is preferentially absorbed by rapidly proliferating cells, such as cancer cells. Finally, [32P]PO4 improves on the previous direct injection of particulate colloidal ³²P into primary tumors, which has met with limited success and has not been shown to prevent or diminish metastases to distant sites.

Secondly, previous regimens of inorganic ³²P for polycythemia vera and essential thrombocythemia used very high doses of ³²P, in the range of 15-30 mCi per patient. In contrast, our doses of inorganic ³²P are at least one order of magnitude lower (equivalent to a human dose of 0.75 to 7.5 mCi). For this reason, in xenografted nude mice as well as in syngeneic BALB/c mouse tumor systems, although our ³²P doses strongly and effectively inhibited tumor growth, there were no apparent detrimental effects in terms of weight gain, general activity, or overall health.

Thirdly, in contrast to other beta-emitting isotopes such as ¹³¹I and ⁹⁰Y, ³²P is incorporated directly into DNA. Our data suggest that this incorporation dramatically increases the cell-killing efficiency of ³²P, since the decay of incorporated ³²P to sulfur chemically breaks the first strand of the DNA and the released electron needs to travel only 2 nm to reach its contralateral DNA strand. Thus, this process efficiently causes double-strand DNA breakage, which is required to overcome innate DNA repair pathways and achieve cell death. In contrast to ³²P, other electron-emitting isotopes (such as ¹³¹I and ⁹⁰Y) emit electrons from distances of 1,000 to 5,000 nm away from DNA, some 500- to 2,500-fold farther than the distance of an incorporated ³²P atom from its sister DNA strand.

It is intriguing to note that early researchers performing Sanger sequencing with [³²P]dATP in the 1980's noted that sequencing products required electrophoresis within two days after the sequencing reaction, otherwise bands seemed to disperse and were difficult to interpret. This same principle may operate with ³²P as an anti-cancer agent. The decay of ³²P to sulfur chemically shears the strand of DNA into which it is incorporated. Without being limited to this theory, we hypothesize that this event, coupled with the extremely close proximity of the incorporated radioisotope to its sister DNA strand, results in a dramatic increase in cell-killing efficiency vs. other beta-particle emitters such as ¹³¹I and ⁹⁰Y, which are not incorporated. The resulting implications for the potential clinical treatment both primary and metastatic human tumors are obvious and inescapable.

### Example 2

### Further Investigation of the To Investigate the Growth-Inhibitory Effects of [³²P]ATP, ³²P-pyro-PO₄, & ³²P-monoPO₄ Against Tumors in Normal BALB/c Mice & in Genetically Engineered Murine Cancer Models

Syngeneic tumors are established in immunocompetent mice. Tumor-inhibitory efficacy of different ³²P chemical forms are compared at various doses and timepoints. [³²P]ATP, ³²P-pyroPO₄, & ³²P-monoPO₄ are tested against tumors in the Min (multiple intestinal neoplasia) and K-ras^{G12D}-mutant mouse models. Synergy of ³²P with nonradioactive agents including, but not limited to, hydroxyurea and cisplatin, are tested in combination therapy experiments.

### Example 3

### To Study Mechanisms Underlying Tumor Inhibition by ³²P

Immunocompetence of normal mice with syngeneic tumors are determined at various timepoints and doses after [³²P]ATP, ³²P-pyroPO₄, and ³²P-monoPO₄. Lymphokine levels are measured, syngeneic tumor and selected organs are histologically examined, and immune cells are analyzed by FACS. Expression levels of genes involved in the establishment and maintenance of the immune system are determined by qRT-PCR. Biodistribution of ³²P to primary and metastatic tumors and major organs are measured. Autoradiography of slide preparations are performed by silver-emulsion development and H&E staining to show which cells and structures in tumors concentrate ³²P & when this uptake occurs. DNA double-strand breaks are quantified by gamma-H2AZ assays, measuring phosphorylation of H2AX histone by ataxia-telangectasia-related (ATR) protein.

1. A method for treating cancer in a patient comprising the step of systemically administering to the patient an effective amount of aqueous ³²P.

2. The method of claim 1, wherein the ³²P is ³²P monophosphate.

3. The method of claim 1, wherein the ³²P is ³²P pyrophosphate.

4. The method of claim 1, wherein the aqueous ³²P is administered intravenously.

5. The method of claim 1, wherein the patient has metastatic cancer.

6. The method of claim 1, wherein the cancer is colon cancer.

7. The method of claim 1, wherein the cancer is lung, breast or pancreatic cancer.

8. The method of claim 1, wherein the ³²P is administered in a low dose amount.

9. The method of claim 1, wherein the ³²P is administered in a dosage range of about 0.5 mCi to about 10.0 mCi.

10. The method of claim 1, wherein the ³²P is administered in a dosage range of about 0.75 mCi to about 7.5 mCi.

11. The method of claim 1, wherein the ³²P is administered in a dose of about 750 μCi.

12. A method for treating cancer in a patient comprising the step of intravenously administering a low dose of aqueous ³²P monophosphate or ³²P pyrophosphate to the patient.

13. The method of claim 12, wherein the patient has metastatic cancer.

14. The method of claim 12, wherein the cancer is colon cancer.

15. The method of claim 12, wherein the cancer is lung, breast or pancreatic cancer.

16. The method of claim 12, wherein the ³²P is administered in a dosage range of about 0.75 mCi to about 7.5 mCi.

17. The method of claim 12, wherein the ³²P is administered in a dose of about 750 μCi.
